SEQUENCE LISTING (6 sequences)

SEQ ID NO: 1 - DNA, 30 bp, Artificial Sequence
  synthetic DNA
aggatggccgcctttcaggctttgatgcaa

SEQ ID NO: 2 - DNA, 30 bp, Artificial Sequence
  synthetic DNA
ttgcatcaaagcctgaaaggcggccatcct

SEQ ID NO: 3 - DNA, 16 bp, Artificial Sequence
  synthetic DNA
atgcgtccggcgtaga

SEQ ID NO: 4 - DNA, 19 bp, Artificial Sequence
  synthetic DNA
tatgctagttattgctcag

SEQ ID NO: 5 - DNA, 30 bp, Artificial Sequence
  synthetic DNA
aggatggccgccttccaggctttgatgcaa

SEQ ID NO: 6 - DNA, 30 bp, Artificial Sequence
  synthetic DNA
ttgcatcaaagcctggaaggcggccatcct

(19) United States
(12) Patent Application Publication
(10) Pub. No.: US 2013/0005582 A1
(43) Pub. Date: Jan. 3, 2013

(54) PREPARATION OF RESTRICTION ENDONUCLEASES IN IVCS USING FRET AND FACS SELECTION

(76) Inventor: Michal Lower, Warsaw (PL)

(21) Appl. No.: 13/498,584

(22) PCT Filed: Sep. 28, 2010
(86) PCT No.: PCT/PL2010/000099

§ 371 Date: Mar. 28, 2012

(30) Foreign Application Priority Data
Sep. 28, 2009 (PL) P-389135

(51) Int. Cl. C40B 10/00; C07H 21/04

(52) U.S. Cl. 506/1; 536/23.1

(57) ABSTRACT

Method of preparation of restriction endonucleases, particularly those exhibiting the desired sequential specificity consists in that a fluorescence-marked DNA probe is used for screening a library of mutants, preferably in IVC format, and/or using other high-performance screening (HTS) technique, which is attained through expression of proteins included in the library of mutants in a cell-free system in the presence and by means of the DNA probe, and proteins thus obtained, resulting from expression of clones from the library, degrade the DNA probe, if their substrate specificity matches the searched one, the degradation of the DNA probe being detected as a disappearance of the FRET phenomenon between fluorescence markers included in the probe, and then microcompartments in which the FRET phenomenon ceases to occur, are separated from the remaining ones using Fluorescence Activated Cell Sorter (FACS) and/or other equipment for HTS analysis, and then DNA coding clones capable of degrading the probe are amplified using polymerase chain reaction (PCR) technique and are used as a basis for construction of the subsequent library of mutants, which is searched during the subsequent round of screening, according to the scheme mentioned above, and the subsequent rounds of screening are carried out until the enzyme having the desired properties is obtained.

The fluorescence-marked DNA probe is characterized in that the markers of the DNA probe are located in a direct vicinity of recognizable sequence by searched restriction enzyme and/or in the vicinity of DNA restriction sites, and between the markers the FRET (Free Radiationless Energy Transfer) phenomenon occurs.

Object of the invention is a method and a DNA probe for preparation of restriction endonucleases, particularly those exhibiting desired sequential specificity.

Restriction enzymes are proteins that cleave DNA molecule within or in the vicinity of a recognizable sequence. The sequence, understood as a definite sequence of nitrogen bases in the DNA molecule, determines whether cleavage of the molecule will occur or not. One restriction enzyme recognizes and cleaves one strictly defined DNA sequence. Restriction endonucleases are widely used research tools in contemporary molecular biology. At present, more than 3700 restriction endonucleases of type II are known, which specifically recognize 274 nucleotide sequences. This means that more than 80% theoretically possible specificities have not been up to now discovered in the nature [10]. Use of restriction endonucleases in genetic engineering and molecular diagnostics of hereditary diseases results in a growing demand for enzymes exhibiting new specificities. At the same time, mechanism of recognition of DNA sequence by enzymes is still poorly recognized which makes it impossible to employ methods of rational design in the field of engineering specificity.

Targeted protein evolution is known, which is widely used for construction enzymes exhibiting new properties [4]. It consists in carrying out several rounds of mutagenesis and clones selection. The most efficient methods of protein evolution are based on in vitro cell-free compartmentalization systems (IVC). In that system, a searched library of mutants of the protein being investigated is expressed by means of synthetic cellular extract in droplets of mineral oil having volumes of several femtolitres. Thanks to such a system, it is possible to detect enzymatic activity of a single protein molecule. During the reaction, a nonfluorescence substrate of the enzyme being investigated is transformed into a fluorescence product. This makes it possible to sort droplets using a fluorescence activated cell sorter (FACS) [6]. From droplets exhibiting proper fluorescence level, DNA is isolated which constitutes a matrix for a subsequent round of mutagenesis and selection. This allows to search through a library of 10¹⁰-10¹² clones during a single experiment. By comparison, classical in vivo screening methods make it possible to search through a library of about 10⁶ clones [8].

Existing systems of searching through libraries of restriction endonucleases having IVC format do not exploit full potential of the method because selection is based on detection of sticky ends [2] or protection of DNA against digestion by accompanying methyltransferase [9], which limits a selection potential of such system. Because of that, up to now it has not been possible to attain complete change of specificity, which constitutes a condition for using new enzymes on a large scale.

In the prior art, there are known probes, fluorescence markers of which are located on opposite endings of a DNA oligonucleotide (short sequence), whereas the sequence is recognized somewhere in the middle. From laws governing the FRET phenomenon it results that such probe must be short enough to obtain a suitable signal level, which in turn causes poor cleavage by enzymes and additionally impairs ability to detect their activity. Because of that, first restriction enzymes poorly ‘notice’ short fragments of DNA in which a measurable FRET level is still observed.

Prior systems of following restriction by means of fluorescence probes are suitable only for kinetic studies, because they generate a signal upon digesting a DNA sequence of ten to twenty nucleotides at any site which makes impossible efficient selection of mutants in a view of selected substrate specificity [5 i 3].

Unexpectedly, during our studies it has been found that combining a DNA probe with IVC screening techniques leads to a method of preparation of restriction enzymes exhibiting new sequencing specificities, not existing in the nature.

The invention refers to a new method of detection of endonucleolytic activity within the desired DNA sequence and to a screening method of library of enzyme variants (mutants) in view of such activity.

The method of the invention consists in applying a fluorescence-marked DNA probe for screening a library of mutants preferably in IVC format and/or in using other High Throughput Screening (HTS) technique, which is achieved through expression of proteins included in the library of mutants in a cell-free system in the presence and by means of the DNA probe, and then proteins thus obtained, resulting from expression of clones from the library, degrade the DNA probe if their specificity towards a substrate matches the searched one. Degradation of the DNA probe is detected as disappearance of FRET phenomenon between fluorescence markers included within the probe. After that, microcompartments in which the FRET phenomenon ceases to occur are separated from the remaining ones using Fluorescence Activated Cell Sorter (FACS) and/or other equipment for I-ITS analysis. Then, DNA coding clones capable of degrading the probe is amplified using Polymerase Chain Reaction (PCR) techniques is used as a basis for construction of the subsequent library of mutants, which is searched in the subsequent round of screening, according to the above-mentioned scheme, and the subsequent rounds of screening are carried out until an enzyme of desired properties is obtained. The library of mutants is a pool of DNA molecules coding various variants of proteins in such a manner that their expression in a cell-free system can occur.

Expression in the cell-free system is achieved in microcompartments, the size of which is adjusted such that a single compartment includes one to at least several clones from the library of mutants.

The DNA probe of the invention is characterized in that markers of the DNA probe are located in a direct vicinity of a sequence recognized by a searched restriction enzyme and/or in the vicinity of DNA restriction sites, and between the markers the FRET (Free Radiationless Energy Transfer) phenomenon occurs.

The state-of-the-art methods are either accurate and very slow—i.e. searching through a library of mutants by a research team takes at least several years, or are sufficiently fast and very rough—i.e. conditions for selection are too mild and too much improper results is qualified to subsequent rounds, and—as a final result—the whole process collapses because of an excess of cases erroneously taken as positive ones. Only a combination of a stringent selection and a possibility of a search through a huge library makes it possible to obtain restriction endonucleases of the desired sequential specificity.

The invention makes it possible to obtain restriction endonucleases having new specificities toward substrates, not existing in the nature. Thanks to it, generating enzymes for molecular diagnostics using RFLP analysis is possible (polymorphism of a length of restriction fragments). RFLP analysis consists in digesting genetic material of a patient with restriction enzymes, and then separation of these fragments on agarose gel. Basing on a gel image, one can find whether a patient is a carrier of a mutation causing a particular genetic disease. The problem is a small number of enzymes recognizing DNA sequences that are significant from a diagnostic point of view. Thanks to possibility of generating such enzymes RFLP analysis can be applied to screening studies in a view of many genetic diseases.

A use of the probe of the invention makes it possible to search efficiently through a sufficiently large library of clones, to find probably restriction endonucleases of the desired specificity. Location of markers in a direct vicinity of a recognizable sequence and/or a DNA cleavage site provides sufficiently stringent conditions for selection to keep at minimum an amount of cases erroneously regarded as recognizing the searched sequence.

The probe of the invention provides a suitable level of selection and is well processed by the enzymes. The probe has markers located near to each other within a large fragment of DNA. The method of the invention is explained below in preferable examples of embodiments.

EXAMPLE 1

### Construction of a Basic Library of Mutants

Restriction endonuclease MvaI, which recognizes CCWGG sequence (W is A or T), was selected as a core for mutagenesis. Amino acids participating in recognizing the DNA sequence: Y213, H223, D224, H225, R209, D207, T68, R230 and T102 were selected as a randomization target. A primary library of mutants was constructed by a method of combinatorial synthesis using ITERATE™ technology supplied by Geneart company. About 10¹² unique clones were obtained. Each of the clones codes a sequence of a mutated gene of endonuclease MvaI under control of a promoter from bacteriophage T7, which makes it possible to express that gene in a cell-free system of protein synthesis.

### Construction of a Probe for Screening the Library

Screening of the library was carried out to find an enzyme of specificity TCAGG not existing in the nature. For this purpose, a DNA oligonucleotide of the nucleotide sequence: AGGATGGCCGCCTTTCAGGCTTTGATGCAA (oligonucleotide 1, [SEQ ID NO: 1]) was designed. Tymine in position 14 was marked with tetramethylenerodamine (TAMRA), whereas tymine in position 21—with fluorescein. These fluorophores constitute a pair between which the FRET phenomenon occurs. Synthesis of a probe was ordered to an external company. At the same time, synthesis of a not-marked DNA oligonucleotide of the sequence complementary to the probe: TTGCATCAAAGCCTGAAAGGCGGCCATCCT (oligonucleotide 2, [SEQ ID NO: 2]) was ordered. The resulting preparations of oligonucleotides 1 and 2 were suspended in a volume of 10 mM TrisHCl pH 8.0 suitable to obtain final concentration of 0.2 M. Then, equal volumes of solutions of oligonucleotides 1 and 2 were mixed together, heated to 95° C. for 5 minutes and cooled to 10° C. at the rate of 0.5° C. per minute. The resulting duplex of oligonucleotides was used as a DNA probe (Probe 1) in subsequent experiments.

### Expression of the Library of Mutants in a Cell-Free System in IVC Format

0.5 ug DNA library was added to 50 ul of mixture for cell-free protein synthesis supplemented with the DNA probe at the final concentration of 0.02 M. The reaction mixture was added to 0.2 ml solution of the composition: 0.5% Triton-X100; 4.5% Span-80. The resulting mixture was emulsified while shaking at 1600 RPM for 5 minutes at 4° C. To the “water in oil” emulsion thus obtained, the subsequent aqueous phase in a form of 0.6 ml 2% Tween 80 in PBS was then added. The resulting mixture was shaken at 800 RPM for 2 minutes. As a result, a mixture of droplets of oil in the aqueous phase was obtained. In each of the droplets, one to several DNA molecules from a library of mutants, ten to twenty molecules of the probe and a set of substances necessary for in vitro translation and transcription to occur, were closed. The mixture was incubated for 3 hours at 37° C., to allow for protein expression and degradation of the DNA probe.

### Detection of Endonucleolytic Activity and Selection of Clones Exhibiting Desired Specificity Using a High-Performance Flow Cytometer

Upon incubating for three hours, the mixture of droplets was put onto the flow cytometer FACSAria II and separated while keeping the following parameters: die diameter 70 um, sorting rate 70000 events per second, wavelength of fluorescence excitation 480 nm, readout of fluorescence signal within the wavelength range 512-522 nm. The sorter selected droplets of the highest fluorescence. Altogether 96 droplets were collected, which were used for PCR reaction. Each of the droplets was placed in a different well of a 96-well polypropylene plate.

### DNA Amplification of Selected Clones Using PCR Reaction

The following DNA oligonucleotides were used as starters for the PCR reaction: T7F of the sequence ATGCGTCCGGCGTAGA and T7R of the sequence TATGCTAGTTATTGCTCAG. Polimerase Pfu Turbo (Staragene) were used for amplification. Each of the droplets was suspended in 5 ul 10 mM TrisHCl pH 8.0. The plate with suspended droplets was centrifuged at 13000 RPM for 15 minutes. The upper phase of oil was removed, whereas the aqueous phase was extracted with 2 ul diethyl ether, which was removed by evaporation under reduced pressure in a SpeedVac-type apparatus. DNA thus purified was suspended in a 10 ul mixture for PCR of the composition: 20 mM TrisHCl pH 8.8; 10 mM (NH₄)₂SO₄; 10 mM KCl; 0.1% (v/v) Triton X-100; 0.1 mg/ml BSA; 0.125 mM each of dNTP; 0.5 uM starter T7F; 0.5 uM starter T7R; 0.5U polimerase Pfu Turbo. The PCR reaction was carried out in a thermocycler using the following program: 95° C.-3 minutes; (95° C.-30 sec.; 45° C. 35 sec; 72° C.-1 minute), while repeating 30 times operations listed in the parentheses; and 72° C.-10 minutes. The resulting DNA was purified from reaction mixtures using a set for DNA purification after enzymatic reactions “Clean-UP” (A&A Biotechnology). In this way, the amplified library of DNA clones coding restriction enzymes of sequential specificity TCAGG was obtained.

### EXAMPLE 2

The present example assumes additional increase of specificity of enzymes obtained during a process of selection through subsequent rounds of the process, in order to eliminate the primary sequential specificity of the enzyme which was a matrix to form the starting library of clones (in this case, the enzyme is restriction endonuclease MvaI of specificity CCWGG, where W is A or T).

The whole process is carried out in the same way as in Example 1 up to obtaining amplified library of 96 clones of coding enzymes of specificity TCAGG. They are used for generating material for the second round of selection by a method error-prone PCR.

### Generating Material for the Second Round Of Selection by the Error-Prone PCR Method

0.5 ng DNA of each of the clones included in the amplified library was mixed. The mixture was used as a matrix in PCR reaction of the following parameters. The following DNA oligonucleotides were used as starters for the PCR: T7F of the sequence ATGCGTCCGGCGTAGA [SEQ ID NO: 3] and T7R of the sequence TATGCTAGTTATTGCTCAG [SEQ ID NO: 4]. Composition of the reaction mixture: 75 mM Tris-HCl (pH 8.8 at 25° C.); 20 mM (NH₄)₂SO₄; 0.01% (v/v) Tween 20; 7mM MgCl₂; 0.5 mM MnCl₂; 1 mM dCTP; 0.2 mM dATP; 1 mM dTTP; 0.2 mM dGTP; 2.5 U recombined polimerase Taq (Fermentas); 10 uM starter T7F; 10 uM starter T7R. PCR reaction was carried out in a thermocycler using the following program: 95° C.-3 minutes; (95° C.-30 sec.; 45° C.-35 sec; 72° C.-2 minutes) while repeating 25 times operations listed in the parentheses; and 72° C.-10 minutes.

Second Round of Selection

In the second round of selection, in addition to the probe 1 of Example 1, an additional DNA probe (Probe 2) was used, including oligonucleotide 3 of the sequence: AGGATGGCCGCCTTCCAGGCTTTGATGCAA [SEQ ID NO: 5] marked at 5′-end with fluorescence colorant Cy5, and at 3′-end—with quencher

BHQ3 and oligonucleotide 4 of the sequence TTGCATCAAAGCCTGGAAGGCGGCCATCCT [SEQ ID NO: 6]. To prepare a functional probe from oligonucleotides 3 and 4, the same method as in case of oligonucleotides 1 and 2 of Example 1 was used.

The material obtained from the error-prone PCR reaction was subject to expression in a IVC system analogously to the material from the primary library of clones in Example 1, the difference being that both probe 1 and probe 2 were present in the reaction mixture. Both the probes were used at the concentration of 0.02 M.

Upon incubating for three hours, the mixture of droplets was put onto a flow cytometer FACSCAria II and separated while keeping the following parameters: die diameter 70 um, sorting rate 8 000 events per second. The cytometer worked in 2 readout channels. In the first channel, readout of a signal of the probe 1 was collected. The wavelength of fluorescence excitation was 480 nm, whereas a readout of a fluorescence signal was carried out within the wavelength range 512-522 nm. In the second channel, a parallel readout of a signal of the probe 2 was made. The wavelength of fluorescence excitation was 635 nm, whereas a readout of a fluorescence signal was carried out within the wavelength range 655-695 nm. The sorter selected droplets of the highest fluorescence within the wavelength range 512-522 nm and, at the same time, of the minimum fluorescence within the wavelength range 655-695. Altogether 20 droplets were collected, which were used for the PCR reaction analogous to that in Example 1. The resulting DNA was used as a matrix for the subsequent error-prone PCR and after selection 10 DNA clones coding restriction endonucleases of sequential specificity TCAGG free from original specificity of enzyme MvaI (CCWGG) were obtained.

### LIST OF STATE-OF-THE-ART PUBLICATIONS

1. Bernath K, Hai M, Mastrobattista E, Griffiths AD, Magdassi S, Tawfik D S. In vitro compartmentalization by double emulsions: sorting and gene enrichment by fluorescence activated cell sorting. Anal Biochem. 2004 Feb. 1; 325(1):151-7
2. Doi N, Kumadaki S, Oishi Y, Matsumura N, Yanagawa H. In vitro selection of restriction endonucleases by in vitro compartmentalization. Nucleic Acids Res. 2004 Jul. 6; 32
3. Eisenschmidt K, Lanio T, Jeltsch A, Pingoud A. A fluorimetric assay for on-line detection of DNA cleavage by restriction endonucleases. J Biotechnol. 2002 Jun. 26; 96(2):185-91
4. Farinas E T, Bulter T, Arnold F H. Directed enzyme evolution. Curr Opin Biotechnol. 2001 December; 12(6):545-51
5. Ghosh S S, Eis P S, Blumeyer K, Fearon K, Millar D P. Real time kinetics of restriction endonuclease cleavage monitored by fluorescence resonance energy transfer. Nucleic Acids Res. 1994 Aug. 11; 22(15):3155-9
6. Griffiths A D, Tawfik D S. Miniaturising the laboratory in emulsion droplets. Trends Biotechnol. 2006 September; 24(9):395-402
7. Kim T W, Keum J W, Oh I S, Choi C Y, Park C G, Kim D M. Simple procedures for the construction of a robust and cost-effective cell-free protein synthesis system. J Biotechnol. 2006 Dec. 1; 126(4):554-61
8. O'Hare H M, Johnsson K. The laboratory in a droplet. Chem. Biol. 2007; 12, 1255-1257
9. Rimseliene R, Maneliene Z, Lubys A, Janulaitis A. Engineering of restriction endonucleases: using methylation activity of the bifunctional endonuclease Eco57I to select the mutant with a novel sequence specificity. J Mol Biol. 2003 Mar. 21; 327(2):383-91
10. Roberts R J, Vincze T, Posfai J, Macelis D. REBASE—enzymes and genes for DNA restriction and modification. Nucleic Acids Res. 2007 January; 35
11. Zheng Y, Roberts R J. Selection of restriction endonucleases using artificial cells. Nucleic Acids Res. 2007; 35

1. A method for the preparation of restriction endonucleases, particularly those exhibiting desired sequential specificity using a DNA probe, method of protein evolution, IVC screening technique, equipment for fluorescence activated cell sorting and technique of polymerase chain reaction characterized in that, the fluorescence-marked DNA probe is used for screening a library of mutants preferably in IVC format and/or using other high-performance screening (HTS) technique, which is carried out through expression of proteins included in the library of mutants in a cell-free system in the presence and by means of the DNA probe, and the proteins thus obtained, which are an effect of expression of clones from the library, degrade the DNA probe, if their specificity towards a substrate matches the searched one, and the DNA probe degradation is detected as disappearance of FRET phenomenon between fluorescence markers included in the probe, and then microcompartments in which the FRET phenomenon ceases to occur, are separated from the remaining ones using Fluorescence Activated Cell Sorter (FACS) and/or other equipment for HTS analysis, and then DNA coding clones capable of degrading the probe are amplified using polymerase chain reaction (PCR) techniques and they are used as a basis for construction of the subsequent library of mutants, which is searched through during the subsequent round of screening, according to the above mentioned scheme, and the subsequent rounds of screening are carried out, until an enzyme of the desired properties is obtained.

2. The method according to claim 1 characterized in that, the expression in a cell-free system is carried out in microcompartments, a size of which is adjusted such, that the single compartment includes one to at least several clones from the library of mutants.

3. A fluorescence-marked DNA probe characterized in that, markers of the DNA probe are located in a direct vicinity of recognizable sequence by the searched restriction enzyme and/or in the vicinity of DNA restriction sites, and between the markers the FRET (Free Radiationless Energy Transfer) phenomenon occurs.
